(19) United States
(12) Patent Application Publication
(10) Pub. No.: US 2007/0232561 A1
(43) Pub. Date: Oct. 4, 2007

(54) Pharmaceutical compositions for promoting wound healing

(76) Inventors: Edward Leung, Cary, NC (US); Kevin H. Sills, Apex, NC (US); Martin W. Beasley, Cary, NC (US)

Correspondence Address: Christopher A. Klein;King Pharmaceuticals, Inc., 400 Crossing Blvd., Bridgewater, NJ, 08807, US

(21) Appl. No.: 11/729,624

(22) Filed: Mar. 29, 2007

Related U.S. Application Data
(60) Provisional application No. 60/788,303, filed on Mar. 31, 2006.

(51) Int. Cl. A61K 31/7076; A61K 9/14

(52) U.S. Cl. 514/47; 424/488

(57) ABSTRACT

The present invention relates to pharmaceutical compositions and methods for promoting wound healing. The invention also relates to methods of making pharmaceutical compositions disclosed herein. Pharmaceutical compositions are disclosed comprising an effective amount of a 2-alkoxyadenosine or 2-aralkoxyadenosine, about 10% to about 70% w/w propylene glycol and a thickening agent.

This application claims benefit of Provisional Application Ser. No. 60/788,303, filed Mar. 31, 2006. The entire contents of the above application is herein incorporated by reference, in its entirety.

### FIELD OF THE INVENTION

The present invention relates to pharmaceutical compositions and methods for promoting wound healing. The invention also relates to methods of making pharmaceutical compositions disclosed herein.

### BACKGROUND

Wound healing is a complex process characterized by three overlapping phases: inflammation, tissue formation, and tissue remodeling. During tissue formation, growth factors synthesized by local and migratory cells stimulate fibroblasts to migrate into the wound where they proliferate and construct an extracellular matrix. Chronic wound healing is characterized by additional complexities and conventional types of therapy are oftentimes inadequate for healing chronic wounds. Indeed chronic wounds resist healing and closure. It is not uncommon that wounds such as diabetic ulcers will become chronic open wounds (Wieman, et al., Diabetes Care, 1998, Vol. 21, No. 5, 822-827).

Current chronic wound care practices include debridement, frequent dressing changes, infection control and a non-weight bearing regimen. Initially debridement which is the removal of necrotic nonviable tissue from the wound site occurs. Removal of the necrotic tissue produces a wound that is now acute rather than chronic. In this way, the body's normal wound healing mechanism can be restarted (Pierce, American Journal of Pathology, 2001, Vol. 159, No. 2, 399-403). Cleansing of the wound of foreign debris and contaminants is critical in chronic wound care. Oftentimes, the dressings of the wound can contribute to such foreign debris and contamination. The presence of this foreign debris contributes to infection of the wound. Although all chronic wounds are colonized by bacteria, when the bacterial burden overwhelms the patient's immune response and the bacteria can grow unchecked, the bacteria will impede any spontaneous healing processes. Indeed, the bacterial burden (also known as biological burden) can contribute to infection and/or inflammation.

Another factor critical to successful chronic wound care is the moisture of the wound environment. Studies have demonstrated that a moist environment promotes re-epithelialization and healing. Understandably, given the numerous factors that contribute to successful healing of chronic wounds, patient compliance with current therapies is low and it is not unusual that a wound remains chronic or reverts to being chronic even after initial successful healing. Moreover, the treatment of non-chronic wounds will often entail meeting many of the same criteria associated with successful chronic wound healing.

Given the complexities associated with successful wound healing, a pharmaceutical drug delivery vehicle useful in promoting wound healing must overcome a number of obstacles in order to be effective. Naturally, the vehicle must be able to carry the active agent prior to application and deliver it to the wound site upon application. Next, the vehicle must be capable of delivering the agent to the wound site without producing unacceptable levels of systemic absorption or permeation in order to avoid systemic pharmacologic action. This problem is particularly acute in chronic wound treatment as the debridement often opens the wound to the vascular system of the body, thereby presenting a potential entrance for systemic absorption or permeation of the active agent. Thus, there is a need for formulations that when used in treatment of patients does not produce unacceptable levels of systemic absorption of the active agent.

Any delivery vehicle must also be bacteriostatic or prevent any bacterial growth. The vehicle must enter the wound as clean as possible. Moreover, a level of cleanliness must be preserved throughout the administration. (Sibbald, et al., Ostomy/Wound Management, 2003, Vol. 49, Issue 11, 24-51). Typically in order to obtain the level of sterility required to prevent the introduction of a bacterial burden, a formulation must be sterilized. A formulation may be rendered sterile by aseptic manufacturing procedures or terminal sterilization. Terminal sterilization usually involves exposure to a radiological or thermal source to achieve the requisite degree of sterilization. However, these methods of terminal sterilization often have deleterious effects and may degrade the active agent or the other components of the composition which in turn decrease the effectiveness of the ultimate pharmaceutical composition (Remington: The Science and Practice of Pharmacy, 21^{st} Ed., Lippincott Williams & Wilkins, Philadelphia, Pa., 2005, 776-777, 794-797). As such, there is a need for formulations that does not need to be sterilized via aseptic manufacturing procedures or terminal sterilization

Diabetes is a major U.S. health concern affecting nearly 6% of the population, or 16 million people. The incidence of diabetes is increasing at a rate of approximately 800,000 new cases diagnosed per year (Centers for Disease Control and Prevention. National Diabetes Fact Sheet: National estimates and general information on diabetes in the United States. Revised edition. Atlanta, Ga.: U.S. Department of Health and Human Services, Centers for Disease Control and Prevention, 1998.). With the increasing prevalence of diabetes, the neuropathic foot ulcer has become a major physical, emotional, and economic burden affecting patients, families, caregivers, and health systems. Approximately 15% of diabetic patients (i.e., 2-3 million patients) will develop foot ulceration during the course of their disease (Reiber GE., Diabet Med 1996;13:S6-S11). Hospital admissions of diabetic patients with foot ulcers are often prolonged by infection, gangrene, and lower extremity amputation; and actually account for more in-hospital days than any other complication of diabetes (Bild, et al., Diabetes Care 1989;12(1):24-31).

After restoring blood flow, if necessary, the cornerstone of therapy for the treatment of neuropathic foot ulcers is the optimization of standard wound care: (1) initial sharp debridement of callous, fibrin and necrotic tissue, followed by additional debridement if indicated, (2) aggressive management of a non-weight-bearing regimen including the use of wheel chairs, crutches, walkers, molded shoes, etc., (3) moist wound dressings, (4) nutritional support and maintaining optimal glycemic control, and (5) infection surveillance and treatment (American Diabetes Association. Consensus development conference on diabetic foot wound care. Diabetes Care 1999;22(8):1354-1360; U.S. Department of Health and Human Services, Food and Drug Administration. Guidance for Industry: Chronic cutaneous ulcer and burn wounds—developing products for treatment. Draft Guidance. June 2000; Steed, et al., J Am Coll Surg 1996; 183:61-64; Frykberg, et al., Diabetic foot disorders: a clinical practice guideline. Data Trace Publishing Company, 2000).

New pharmacologic agents and devices recently have been introduced for the management of chronic wounds. These agents should be considered adjunctive to standard care. Growth factors such as platelet-derived growth factor have been shown to be modestly effective (Steed, et al., J Vasc Surg 1995;21:71-81; Wieman, et al., Am J Surg 1998;176(2A):74S-79S; Wieman, et al., Diabetes Care 1998;21(5):822-827). However, high concentrations of elastases, collagenases, and other proteases in the extracellular matrix of chronic wounds could eradicate the beneficial cytokines and cytokine receptors present in the wound bed, making them less effective (Wysocki, J WOCN 1996;23:283-290; Mast, et al., Wound Rep Reg 1996;4:411-420; Yager, et al., J Invest Dermatol 1996;107:743-748; Yager, et al., Wound Rep Reg 1997;5:23-32). Furthermore, leakage and accumulation of macromolecules such as fibrin, α₂ macroglobulin, and albumin in the wound bed may trap growth factors making them unavailable to the tissue and to the wound (Falanga, et al., Lancet 1993;341:1006-1008). Skin substitutes also have shown evidence of efficacy (Falanga, et al., Wound Rep Reg 1999;7:201-207; Veves, et al., Diabetes Care 2001;24(2):290-295; Gentzkow, et al., Diabetes Care 1996;19(4):350-354; Bowering, J., Cutan Med Surg 1998;3(Suppl 1):S1-29-32), but also can be destroyed by proteases for the reasons noted previously. Commercial growth factors and skin substitutes are expensive agents that make cost-effectiveness an issue for many patients and third-party payers.

As noted above, there are currently few therapies available that meet all of the criteria necessary for successful wound healing and furthermore, therapies have not been effective in promoting and achieving successful chronic wound healing and closure. As such, there is a need for drug delivery vehicles capable of delivering wound healing active agents, in particular those that are capable of facilitating chronic wound healing and closure. Moreover, there is a need for drug delivery vehicles that can achieve many of the criteria necessary for successful healing of wounds, particularly chronic wounds.

Because in vitro studies demonstrate that adenosine A₂ receptor agonists promote fibroblast and endothelial cell migration into an artificial wound (Montisinos, et al., J. Exp. Med. Nov. 3, 1997; 186(9):1615-20), it is of particular interest to develop pharmaceutical compositions that contain A₂ receptor agonists useful in wound healing.

U.S. Pat. No. 6,020,321, issued Feb. 1, 2000 to Cronstein, et al. discloses topical preparations of a number of adenosine A₂ agonists in an ointment base such as PEG-1000. The topical application of agonists of the adenosine A₂ receptor increases endothelial cell and fibroblast migration, key factors of wound healing. Examples of such agonists are 2-phenylaminoadenosine, 2-para-2-carboxyethylphenyl-amino-5′-N-ethylcarboxamido-adenosine, 5′-N-ethylcarboxamidoadenosine, 5′-N-cyclopropyladenosine, 5′-N-methyl-carboxamidoadenosine and PD-125944 (PCT International Publication No. WO 94/23723). Likewise, adenosine A₂ agonist CGS 21680 (2-[p-(carboxyethyl)phenylethylamino]-5′-N-ethylcarboxamidoadenosine) has been shown to increase the rate of wound closure in rats (Monetesinos, et al., American Journal of Pathology, Vol. 160, No. 6, 2002, 2009-2018).

U.S. Pat. No. 6,951,932 issued Oct. 4, 2005 to Moorman discloses the synthesis of 2-aralkoxy and 2-alkoxy adenosine derivatives, specifically 2-[2-(4-chlorophenyl) ethoxy]adenosine. 2-[2-(4-Chlorophenyl)ethoxy]adenosine has been found to promote wound healing in healthy BALB/C mice (Victor-Vega, et al., Inflammation, 2002, February; 26(1):19-24).

Notwithstanding the recognition that adenosine A₂ receptor agonists can promote wound healing, there remains the need for pharmaceutical delivery vehicles, especially stabilized pharmaceutical delivery vehicles, that are able to deliver these agents in an effective amount to the wound site and meet the various criteria essential to successful wound healing. Indeed, to date, there is no pharmaceutical composition with an adenosine A₂ receptor agonist with a demonstrated efficacy in wound healing.

### SUMMARY OF THE INVENTION

The subject invention relates to a pharmaceutical composition containing adenosine A₂ receptor agonists useful in promoting wound healing, including chronic wounds. Specifically, the subject invention relates to pharmaceutical composition useful in promoting wound healing, including chronic wounds containing 2-alkoxyadenosine or 2-aralkoxyadenosine derivatives. In particular, applicants have discovered that a pharmaceutical composition comprising 0.5-500 μg/g of 2-[2-(4-chlorophenyl)ethoxy]adenosine in 50% w/w propylene glycol is effective to promote chronic wound healing and closure without systemic absorption of the active agent into the body and without introducing an increased biological burden to the wound site. As such, the instant application relates to a pharmaceutical composition comprising a wound healing agent in a glycol, especially in a propylene glycol, drug delivery vehicle wherein the pharmaceutical composition can be used for the treatment of all wound types, acute or chronic, such that the wound undergoes healing more rapidly than similar wounds left to heal naturally or which are treated with currently available methods. Applicants have also found that unlike current wound healing therapies, the pharmaceutical compositions of the invention have bacteriostatic antimicrobial properties and can be prepared without the use of conventional sterilization methods. As such, the pharmaceutical compositions of the instant invention can be readily manufactured at a lower cost in comparison to current wound healing therapies that require the utilization of sterilization methods.

The instant invention encompasses pharmaceutical compositions that are stable over time and do not exhibit significant amounts of degradant products of the active pharmaceutical substance in the formulation. It is preferred that the amount of degradant products of the active pharmaceutical substance in the formulation is, in total, less than 5% at the up to about 36 months. It is preferred that the amount of degradant products of the active pharmaceutical substance in the formulation is, in total, less than 5% up to about 4 ½ years.

The instant invention encompasses pharmaceutical compositions that allow for minimal, substantially none, or no systemic absorption of the active agent into the body outside of the wound site when administered to a patient. The instant invention encompasses pharmaceutical compositions that allow for minimal, substantially none, or no systemic absorption as shown by minimal, substantially none, or non detectable levels of the active agent into the blood plasma of the patient. The instant invention also encompasses pharmaceutical compositions that are self-preserving and undergo very little degradation. Moreover, pharmaceutical compositions of the instant invention need not be conventionally sterilized by irradiation or heat in order to avoid the introduction of an additional biological burden into the wound site.

The present invention also encompasses methods for promoting wound healing in a patient, comprising administering to said patient an amount of a pharmaceutical composition of the invention effective to promote wound healing.

Administering a pharmaceutical composition of the invention results in minimal levels of active agent in the blood of the patient. The methods of the invention comprise administering the pharmaceutical compositions of the invention which introduce no additional biological burden into the wound site and do not cause a systemic pharmacological reaction such as flushing, or increased heart rate, as might be expected from the systemic administration of an adenosine A_{2A} receptor agonist.

The pharmaceutical compositions and methods of the invention can be employed to promote wound healing in a wide variety of wounds. The wound can be the result of, but not limited to, venous leg ulcers, pressure ulcers, diabetic neuropathic ulcers, burn injuries, surgical wounds, acute wounds and other dermatological conditions that interfere with the integrity of the skin, and are caused by pharmacologic/pathologic mechanisms treated by the invention. The instant invention also envisions that the pharmaceutical compositions and methods of the invention are capable of delivering an amount of an active agent to a chronic open wound effective to promote wound healing and closure.

The subject invention further contemplates impregnating the bandages, wound protective dressings, foams, sponges, pads, gauzes, collagen, film dressings, drapes or pastes with the pharmaceutical composition for use in the treatment of wounds.

The present invention also contemplates kits comprising the pharmaceutical compositions of the invention effective to promote wound healing. Kits may include but are not limited to bandages, wound protective dressings, foams, sponges, pads, gauzes, collagen, film dressings, drapes and pastes, optionally incorporating a pharmaceutical composition of the invention.

### DETAILED DESCRIPTION

A. Pharmaceutical Compositions

The instant invention encompasses pharmaceutical compositions comprising an effective amount of an active agent in a drug delivery vehicle useful in the treatment of wounds. Preferably, the pharmaceutical composition is a gel. In certain embodiments, the pharmaceutical composition is a gel, cream, an ointment, or a lotion. Preferably the pharmaceutical compositions of the invention are administered topically. It is preferable that the pharmaceutical composition does not irritate or cause pain or inflammation with respect to the wound due to the composition's excipients.

The instant invention encompasses pharmaceutical compositions that comprise about 10% to about 70% w/w glycol, preferably about 20% to about 60% w/w glycol, most preferably about 50% w/w glycol. In various embodiments the pharmaceutical compositions are 10% to 70% w/w glycol, preferably 20% to 60% w/w glycol, most preferably 50% w/w glycol. In accordance with the present invention the glycol may be a C1 to C9 alkyl diol or the polymer of the diol. In a preferred embodiment, the glycol is propylene glycol.

In each of the above embodiments, the pharmaceutical composition can further comprise a thickening agent. Examples of thickening agents useful in the subject invention include but are not limited to acacia, alginic acid, bentonite, carbomer, carboxymethylcellulose sodium, cetostearyl alcohol, colloidal silicon dioxide, ethylcellulose, gelatin, guar gum, hydroxyethyl-cellulose, hydroxypropyl cellulose, hydroxypropyl methylcellulose, magnesium aluminum silicate, maltodextrin, methylcellulose, polyvinyl alcohol, povidone, propylene carbonate, propylene glycol alginate, sodium alginate, sodium starch glycolate, starch, tragacanth, and xanthan gum. In an embodiment of the subject invention, the thickening agent is a cellulose. In a preferred embodiment of the subject invention, the microcrystalline cellulose is sodium carboxymethylcellulose.

Preferably, the drug delivery vehicle delivers an amount of an active agent locally to a wound effective to promote wound healing and closure with minimal, substantially none, or no systemic absorption of the active agent into the body aw ay from the wound. Systemic absorption may be tested for by examining the levels of active agent in the blood plama of the subject, as described further herein.

In preferred embodiments, the vehicle has antimicrobial properties. In other preferred embodiments, the vehicle prevents degradation of the active agent. In yet other preferred embodiments, the vehicle does not introduce a biological burden into the wound site. As disclosed herein, the vehicle according to the present invention does not require sterilization using conventional sterilization methods, including but not limited to thermal or irradiation methods.

As such, the instant invention also contemplates pharmaceutical compositions that have antimicrobial properties. In preferred embodiments, the pharmaceutical compositions are not susceptible to degradation. Moreover, in other preferred embodiments, the pharmaceutical compositions do not introduce a biological burden into the wound site. In certain preferred embodiments, the pharmaceutical composition need not be manufactured under aseptic conditions or packaged into sterilized packaging.

In certain embodiments, the pharmaceutical compositions comprise an effective amount of an adenosine A₂ receptor agonist. Examples of adenosine A₂ receptor agonists useful in the instant invention include but are not limited to 2-phenylaminoadenosine, 2-(para-(2-carboxyethyl)phenyl)-amino-5′-N-ethylcarboxamido-adenosine, 5′-N-ethylcarboxamido adenosine, 5′-N-cyclopropyladenosine, 5′-N-methyl-carboxamidoadenosine (CGS-21680) and 2-[6-[2-(3,5-dimethoxyphenyl)-2-(2-methylphenyl)-ethyl]aminopurin-9-yl]-5-(hydroxymethyl) oxolane-3,4-diol (PD-125944). In preferred embodiments, the adenosine A₂ receptor agonist is a 2-alkoxyadenosine or 2-aralkoxyadenosine. In such embodiments, the 2-alkoxyadenosine or 2-aralkoxyadenosine is a 2-aralkoxyadenosine. In a particularly preferred embodiment, the 2-alkoxyadenosine or 2-aralkoxyadenosine is 2-[2-(4-chlorophenyl)ethoxy]adenosine. U.S. Pat. No. 6,951,932 issued Oct. 4, 2005 to Moorman discloses the synthesis of 2-aralkoxy and 2-alkoxy adenosine derivatives, specifically 2-[2-(4-chlorophenyl) ethoxy]adenosine.

In pharmaceutical compositions of the invention, the amount of the adenosine A₂ receptor agonist is about 0.1 μg/g to about 1000 μg/g of the pharmaceutical composition. In another embodiment, the amount of the adenosine A₂ receptor agonist is about 0.1 μg/g to about 600 μg/g, about 0.5 μg/g to about 10 μg/g, about 10 μg/g to about 100 μg/g or about 100 μg/g to about 600 μg/g of the pharmaceutical composition. In preferred embodiments of the subject invention, the amount of A₂ receptor agonist is about 0.5 μg/g, 5 μg/g, 20 μg/g, 50 μg/g, 100 μg/g or 500 μg/g of the pharmaceutical composition.

In pharmaceutical compositions of the subject invention, the amount of adenosine A₂ receptor agonist is about 0.00001 to about 0.10% w/w of the pharmaceutical composition. In another embodiment, the amount of the adenosine A₂ receptor agonist is about 0.00001 to about 0.0010% w/w, about 0.0010 to about 0,010% w/w or about 0.01 to about 0.10% w/w of the pharmaceutical composition. In the most preferred embodiments, the amount of A₂ receptor agonist is 0.00005, 0.0005, 0.005, or 0.05% w/w of the pharmaceutical composition.

In a particularly preferred embodiment, the pharmaceutical composition comprises 5 μg/g 2-[2-(4-chlorophenyl)ethoxy]adenosine and 50% w/w propylene glycol. In another particularly preferred embodiment, the pharmaceutical composition comprises 20 μg/g 2-[2-(4-chlorophenyl)ethoxy]adenosine and 50% w/w propylene glycol. In yet another particularly preferred embodiment, the pharmaceutical composition comprises 50 μg/g 2-[2-(4-chlorophenyl)ethoxy]adenosine and 50% w/w propylene glycol. In yet another particularly preferred embodiment, the pharmaceutical composition comprises 100 μg/g 2-[2-(4-chlorophenyl)ethoxy]adenosine and 50% w/w propylene glycol. In yet another particularly preferred embodiment, the pharmaceutical composition comprises 500 μg/g 2-[2-(4-chlorophenyl)ethoxy]adenosine and 50% w/w propylene glycol.

In other embodiments of the subject invention, the pharmaceutical compositions further comprise an isotonic agent. Examples of pharmaceutically acceptable isotonic agents include, but are not limited to, sodium chloride, dextrose, and calcium chloride. In certain embodiments, the isotonic agent comprises a salt, preferably sodium chloride.

In yet other embodiments of the subject invention, the pharmaceutical compositions further comprise water. In yet another embodiment of the subject invention, the water is about 30% to about 90% w/w of the composition.

In other embodiments, the pharmaceutical compositions further comprise a buffering system. Examples of pharmaceutically acceptable buffering systems include, but are not limited to, acetic, benzoic, ascorbic, carbonic, gluratic, malic, succinic, tartaric, citric, and phosphoric. In a preferred embodiment, the buffering system is an acetic system.

In yet another embodiment of the subject invention, the pH of the pharmaceutical composition is from about 4.5 to about 11.0. In preferred embodiments of the subject invention, the pH of the pharmaceutical composition is from about 5.5 to about 10.0. It is further preferred that the pH is from about about 5.9 to about 6.7. In the most preferred embodiment of the subject invention, the pH of the pharmaceutical composition is about 6.5.

In a preferred embodiment, additional preservatives are absent in the pharmaceutical compositions of the invention. In alternate embodiments, additional preservatives including, but not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol and thimerosal can be added to the pharmaceutical compositions of the instant invention.

In an embodiment of the subject invention, the pharmaceutical composition has a viscosity level such that the composition has adequate substantivity to be applied and adhere topically to wounds. In another embodiment of the subject invention, the pharmaceutical composition contains a thickening agent present in sufficient amount to bring the viscosity to a level that will allow for accurate application and adherence to the wound. The shear viscosity of a 1% solution of a pharmaceutically acceptable thickening agent should range from 200 to 2500 cPs when measured with a Brookfield LVF at 30 rpm with either spindle #2 or 3. The setting viscosity of the pharmaceutical composition of the subject invention should be greater than 10,000 cPs when the viscosity is measured using a Brookfield viscometer with small sample cup adaptor at 0.1 rpm with spindle #29 or other appropriate spindle. In a preferred embodiment, the pharmaceutical composition will have an apparent viscosity greater than 100,000 cPs, and in the most preferred embodiment, the pharmaceutical composition will have a viscosity greater than 700,000 cPs.

The instant invention encompasses pharmaceutical compositions that are stable over time and do not during their shelf-life exhibit significant amounts of degradant products of the active pharmaceutical substance in the formulation. It is preferred that the amount of degradant products of the active pharmaceutical substance in the formulation is, in total, less than about 1, 2,3, 4, or 5% at the end of the shelf-life of the product, where the shelf-life is 36 months. It is preferred that the amount of degradant products of the active pharmaceutical substance in the formulation is, in total, less than about 1, 2,3, 4, or 5% at the end of the shelf-life of the product, where the shelf-life is up to 4½ years. In a preferred embodiment the formulation does not does not exhibit detectable amounts of degradants of the active substance within a 3, 6, 9, 12, 18, 24, or 36 month time span when held at 25° C./60% RH. In particular, a very preferred embodiment is a formulation containing 2-[2-(4-chlorophenyl)ethoxy]adenosine that exhibits not more than about 1, 2, 3, 4, or 5% in total, of adenine, adenosine, isoguanosine and 2-(4-chlorophenyl)ethanol) within a 3, 6, 9, 12, 18, 24, or 36 month time span when held at 25° C./60% RH. In particular, a very preferred embodiment is a formulation containing 2-[2-(4-chlorophenyl)ethoxy]adenosine that does not does not exhibit detectable amounts of adenine, adenosine, isoguanosine and 2-(4-chlorophenyl)ethanol) within a 3, 6, 9, 12, 18, 24, or 36 month time span when held at 25° C./60% RH. In particular, a very preferred embodiment is a formulation containing 2-[2-(4-chlorophenyl)ethoxy]adenosine that does not does not exhibit detectable amounts of adenine, adenosine, isoguanosine and 2-(4-chlorophenyl)ethanol) for up to a 6 month time span when held at 40° C./75% RH.

The present invention is directed to pharmaceutical formulations containing 2-alkoxyadenosine or 2-aralkoxyadenosine derivatives, and in a preferred embodiment contains 2-[2-(4-chlorophenyl) ethoxy]adenosine, which maintain at least 60% of their initial potency for a period of up to 3 years. In preferred embodiments the formulations maintain at least 65%, 70%, 75%, 80%, 85%, 90%, 95%, of their initial potency for a period of up to three years. The invention is particularly directed to formulations that maintain at least 80%, 85%, 90%, or 95% of their initial potency after 3 months, at least 70%, 80%, 85%, or 90%, 95% of their initial potency after 6 months, at least 70%, 80%, 85%, 90%, or 95% of their initial potency after 9 months, at least 70%, 80%, 85%, 90%, or 95% % of their initial potency after 12 months, and at least 60%, 70%, 80%, 85%, 90%, or 95% % of their initial potency after 18 months. The present invention is also directed to stabilized 2-[2-(4-chlorophenyl)ethoxy]adenosine formulations that maintain at least 70%, 75%, 80%, 85%, 90%, or 95% of initial label claim potency for a period of two years. The present invention is also directed to stabilized 2-[2-(4-chlorophenyl)ethoxy]adenosine formulations that maintain at least 70%, 75%, 80%, 85%, 90%, or 95% of initial label claim potency for a period of up to three years. The invention is directed to formulations that maintain at least 70%, 75%, 80%, 85%, 90%, or 95% of their initial label potency after 3 months, at least 70%, 75%, 80%, 85%, 90%, or 95% of their initial label potency after 6 months, at least 70%, 75%, 80%, 85%, 90%, or 95% of their initial label potency after 9 months, at least 70%, 75%, 80%, 85%, 90%, or 95% of their initial label potency after 12 months, at least 70%, 75%, 80%, 85%, 90%, or 95% of their initial label potency after 18 months, at least 70%, 75%, 80%, 85%, 90%, or 95% of their initial label potency after 24 months, at least 70%, 75%, 80%, 85%, 90%, or 95% of their initial label potency after 36 months.

B. Administration

The instant invention contemplates methods for promoting wound healing comprising administering the pharmaceutical compositions of the invention to a patient. The methods of the instant invention encompass methods of administering the pharmaceutical compositions of the instant invention whereby the active agent is minimally, substantially not, or not detectable in the blood of the patient administered said pharmaceutical composition. The preferred methods result in minimal, substantially none, or no systemic absorption of the active agent from the pharmaceutical compositions into the body and away from the wound site. Moreover the preferred methods do not introduce an increased biological burden to the wound site.

The subject invention encompasses methods of treating wounds on a patient comprising administration of a pharmaceutical composition of the subject invention. In an embodiment, the patient is a mammal. In a preferred embodiment, the patient is a human. In certain embodiments, the wound is a chronic wound. In preferred embodiments, the chronic wound is a diabetic ulcer. In alternative embodiments, the chronic wound includes but is not limited to venous leg ulcers, pressure ulcers, diabetic neuropathic ulcers, burn injuries, surgical wounds, acute wounds; and other dermatological conditions that interfere with the integrity of the skin, and wounds caused by pharmacologic/pathologic mechanisms treated by the invention.

In a preferred embodiment, the pharmaceutical compositions of the invention are administered topically once a day. Preferably, the patient is administered an amount of about 0.1 μg/day to about 2000 μg/day. Preferably, the patient is administered an amount of about 0.1 μg/day to about 1500 μg/day. Preferably, the patient is administered an amount of about 0.1 μg/day to about 1000 μg/day. Preferably, the patient is administered an amount of about 0.1 μg/day to about 500, 50 or 5 μg/day. In an additional embodiment, a sterile applicator swab is used to apply a thin, uniform film (approximately the thickness of a dime) of the invention in a concentration ranging from 5 μg/g to 500 μg/g over the entire surface area of the wound. In other embodiments, the concentration is from about 0.1 μg/g to about 600 μg/g, about 0.5 μg/g to about 10 μg/g, about 10 μg/g to about 100 μg/g or about 100 μg/g to about 600 μg/g. In preferred embodiments of the subject invention, the concentration is about 0.5 μg/g, 5 μg/g, 20 μg/g , 50 μg/g, 100 μg/g, or 500 μg/g. In a preferred embodiment, the wound is covered with an appropriate dressing.

The methods of the invention result in wound closure achieved in a shorter amount of time as compared to patients treated with currently available wound therapies. The time can be shortened by ¼, ⅓, ½, ¾ of the time for currently available wound therapies. The methods of the invention also result in a greater percentage of wound healing as compared to the percentage of wound healing utilizing currently available wound therapies such as debridement and moist dressings alone. The percentage of wound healing can be 5, 10, 20, 30, 40, 50, 60, 70, 80, 90, or 95 percent greater in a preferred embodiment.

According to the invention, wound healing (e.g., complete epithelialization with no exudate) is assessed clinically by a physician. Alternatively, wound healing is assessed objectively by wound planimetry, measuring wound depth, and photographing the wound.

C. Methods of Preparation

The subject invention also contemplates methods of making the pharmaceutical compositions of the subject invention comprising
a) combining the active agent with the vehicle to produce a solution, and
b) mixing the solution of step a) with a thickening agent, to produce the pharmaceutical composition.

In another embodiment of the subject invention, the method further comprises adding water to the product of step b).

In another embodiment of the subject invention, the method further comprises adding an agent to modify the tonicity of the product of step b) to produce the pharmaceutical composition.

In another embodiment of the subject invention, the method further comprises adding a buffer system to the product of step b) to produce the pharmaceutical composition.

The subject invention also contemplates methods of making the pharmaceutical compositions of the subject invention comprising combining the active agent with the vehicle. In another embodiment, the contemplated method further comprises combining the mixture of the agent and the vehicle with a thickening agent. In yet another embodiment of the invention, the active agent, vehicle and thickening agent mixture can be further combined with an aqueous mixture. In an additional embodiment, the aqueous mixture comprises a buffer system. In the above embodiments, the vehicle can comprise a glycol, preferably propylene glycol.

Preferably, the methods of making a pharmaceutical composition disclosed herein do not include sterilization by gamma irradiation or thermal processes.

Another embodiment of the subject invention is a container comprising a single dose of the pharmaceutical composition of the subject invention. Yet another embodiment of the subject invention is a container comprising multiple doses of the pharmaceutical composition of the subject invention. In the above embodiments, the container comprises an amount of the pharmaceutical composition sufficient for a daily application to a single wound or to multiple wounds. In another embodiment, the container comprises an amount of the pharmaceutical composition sufficient for multiple daily applications to a single wound or to multiple wounds.

The subject invention contemplates impregnating the bandages, wound protective dressing, foams, sponges, pads, gauzes, collagen, film dressings, drapes or pastes with the pharmaceutical composition of the subject invention to be used in the treatment of wounds.

The subject invention also contemplates a kit comprising one or more single dosages of the pharmaceutical composition of the subject invention useful in wound healing. Kits may include but are not limited to bandages, wound protective dressing, foams, sponges, pads, gauzes, collagen, film dressings, drapes and pastes, optionally incorporating a pharmaceutical composition of the invention. The subject invention further contemplates impregnating the bandages, wound protective dressing, foams, sponges, pads, gauzes, collagen, film dressings, drapes or pastes with the pharmaceutical composition of the subject invention.

The subject invention also contemplates a stabilized pharmaceutical composition comprising an effective amount of 2-[2-(4-chlorophenyl)ethoxy]adenosine where the pharmaceutical composition is stable for up to 24 or 36 months. The subject invention contemplates a pharmaceutical composition comprising an effective amount of 2-alkoxyadenosine or 2-aralkoxyadenosine wherein the 2-alkoxyadenosine or 2-aralkoxyadenosine is not systemically absorbed when administered to a patient. The subject invention contemplates a pharmaceutical composition comprising an effective amount of 2-[2-(4-chlorophenyl)ethoxy]adenosine wherein the 2-[2-(4-chlorophenyl)ethoxy]adenosine is not systemically absorbed when administered to a patient. The subject invention contemplates a pharmaceutical composition comprising an effective amount of 2-[2-(4-chlorophenyl)ethoxy]adenosine wherein the 2-[2-(4-chlorophenyl)ethoxy]adenosine is not systemically absorbed when administered to a patient as measured by levels of 2-[2-(4-chlorophenyl)ethoxy]adenosine in the blood plasma levels of the patient. The subject invention contemplates a pharmaceutical composition comprising an effective amount of 2-[2-(4-chlorophenyl)ethoxy]adenosine wherein the 2-[2-(4-chlorophenyl)ethoxy]adenosine is minimally systemically absorbed when administered to a patient. The subject invention contemplates a pharmaceutical composition that is self preserving for up to 12, 24 or 36 months. The subject invention contemplates a pharmaceutical composition that is antimicrobially effective for up to 12, 24, or 36 months.

The subject invention contemplates the use of the formulations of the instant invention with other standard of care methods of wound healing where appropriate. One such non-limiting example is use of the instant formulations in conjunction with vacuum assisted closure (VAC) therapy. VAC therapy, and its use in patients is within the knowledge of one of skill in the art. VAC therapy is an adjunctive therapy system that uses controlled negative pressure (vacuum) to help promote wound healing by removing fluid from open wounds through a sealed dressing and tubing which is connected to a collection container. A non-limiting example of VAC therapy is when wound dressings are made of sterile open-cell foam which is cut to size and placed into or onto the wound bed. The wound site is then covered with an adhesive plastic sheet. The practitioner makes a small hole in the centre of the plastic sheet and the tubing is connected to the sheet, over the hole, by a small plastic dressing. The further end of the tubing is then connected to the VAC pump. Continuous or intermittent sub atmospheric suction pressure of approximately 125 mmHg is then applied to the wound site; although this is adapted according to the individual's needs. Special dressing drapes can be obtained for difficult areas (such as the foot) and new adhesive strips also assist with maintaining an airtight seal.

The term “about” or “approximately” means within an acceptable error range for the particular value as determined by one of ordinary skill in the art, which will depend in part on how the value is measured or determined, i.e., the limitations of the measurement system. For example, about can mean within 1 or more than 1 standard deviations, per the practice in the art. Alternatively, about can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “mammal”, “subject” or “patient” are used interchangeably, unless described as otherwise in the examples, and include, but are not limited to, humans, dogs, cats, horses, pigs, cows, monkeys, rabbits, mice and laboratory animals. The preferred mammals are humans.

The term “self preserving” relates to the ability of a formulation to maintain over time the formulation with no or very low microbial growth and to, if contaminated, reduce the contaminant microbial growth. In particular, it means that the tested formulation meets AET testing, sterility testing and microbial limits testing. In addition, samples which fail such tests at one time point but meet the requirements at a later time point are considered self-preserving.

Antimicrobial Effectiveness Test (AET). The antimicrobial effectiveness test is used to test the effectiveness a pharmaceutical formulation's antimicrobial protection and elucidate if the formulation possesses an intrinsic antimicrobial activity. The test challenges a sample of the pharmaceutical formulation with an inoculum of bacteria or mold which is then tested at several time points for an increase in microbial level. A non-limiting example of such type of testing is described in United States Pharmacopoeia, Chapter 51 (antimicrobial effectiveness testing). In such a test, samples of the pharmaceutical formulation were tested against each of the following E. coli (ACCT. No. 10231), P. aeruginosa (ACTT No. 9027), S. aureus (ACCT No. 6538), C. albicans (ACCT No. 10231) and A. niger (ATCC No. 16404). The viable micro organisms used in the creation of the inoculum must not be more than five passages removed from the original ACTT culture. Suitable media for growing E. coli, P. aeruginosa, and S. aureus soybean-casein digest broth or agar and for C. albicans and A. niger is sabouraud dextrose broth or agar. To make the incoculum, sterile saline TS (or sterile saline ST and 0.05% polysorbate 80 for A. niger) was used to wash the surface growth into a collection vessel. Then inoculum for each bacteria or mold was prepared such that it had a concentration of 1×10⁸ cfu per mL in sterile saline ST (or sterile saline ST and 0.05% polysorbate 80 for A. niger).

Using sterile techniques, the pharmaceutical formulation samples were inoculated with the inoculum of a mold or bacteria and mixed such that the final concentration of cfu in the preparation is between 1×10⁵ and 1×10⁶ cfu per mL of sample. The volume of the suspension innoculum used was between 0.5% and 1.0% of the volume of the sample. The initial concentration of viable microorganisms in each test preparation was estimated based upon the concentration of microorganisms in each of the standardized inoculum as determined by the plate count method. The test samples are incubated at 22.5° C.±2.5° C. along with suitable controls and sampled at set time points. The number of cfu at each time point is determined by the plate count method. The calculated concentrations of cfu per mL present at the start of the test is used to determine the change in log₁₀ values of the concentration of cfu per mL for each microorganism at the applicable test interval which are expressed in terms of log reductions. A sample is considered to be antimicrobially effective (conforms) if with bacteria there is not less than 2.0 log reduction from the initial count at 14 days, and no increase from the 14 days' count at 28 days and with yeasts and molds there is no increase from the initial calculated count at 14 and 28 days.

Sterility Testing. A formulation may be tested for its sterility with respect to bacteria and molds. A non-limiting example of such is test is described in United States Pharmacopoeia, Chapter 71. An example of testing done on the formulations of the current invention is werethe testing was done in a clean room, under a laminar flow hood with use of proper aseptic techniques and disinfectants. One 250 mL tube of sterile fluid thioglycollate media (FTM), one 250 mL tube of sterile Trypticase soy broth media (TSB), and one 15×150 mm Trypticase Soy agar plate (cover off) are held in the laminar hood as environmental air controls. The environmental controls are incubated in the same manner as the tested samples. One 250 mL tube of sterile fluid thioglycollate media and one 250 mL tube of sterile Trypticase soy broth media is each inoculated with 2 g of sample. The TSB tube is incubated at 22.5±2.5° C. and the FTM tube is incubated at 32.5±2.5° C. for a minimum of 14 days. Negative controls using similar TSB and FTM tubes, which are not inoculated by sample, were prepared and incubated in the same manner. In addition, aliquots of the culture media (before placed into the tubes) was also incubated as a negative control. The test and control samples were then observed at the macroscopic level for evidence of microbial growth such as the development of turbidity, sedimentation or surface growth (pellicle formation). If no evidence of microbial growth is found the sample is considered to meet the test for sterility. If there is microbial growth detected the samples is them speciated against particular bacteria and molds. In particular S. aureus (ATCC 6538), Ps. Aeruginosa (ATCC 9027), C. sporogenes (ACCC No. 11437), B. subtili (ATCC 6633), C. albicans (ATCC 10231), A. niger (ATCC 16404).

Microbial Testing: A formulation may be tested for the amount of microbial growth in the sample. A non-limiting example of such a test is described in United States Pharmacopoeia, Chapter 61 (Microbiological Examination of Non-Sterile Products: Microbial Enumeration Test). The microbial enumeration test is a basic, simple design to count the number of CFU in a product or material either as a total count of all microorganisms that create the CFU or as the CFU count of specific microorganisms. The preferred method is to put the material into solution and then plate aliquots to determine the CFU/gram (or mL) of initial material. The method of plating can be either pour plate, spread plate or the filtration of material and then placing the membrane filter on the surface of an agar plate.

Samples of the pharmaceutical formulation were tested against bacteria, yeast, molds. Two parameters were included in this testing (1) total plate count cfu (2) cfu of E. coli, Salmonella, P. aeruginosa, S. aureus. The pharmaceutical formulation samples to be tested were prepared by placing 10 g of the sample into 100 mL of the media of tryptase soy broth (TSB)+Lecithin (or 1 g into 10 ml of media). It was then stirred to form a homogeneous suspension/solution. TSB was used as an enrichment media for microbes. Lecithin was a neutralizer used to deactivate any antimicrobial effects which may be possessed by the sample. The sample was diluted to 1/10 with the appropriate media. (If the test results from the 1/10 dilution proved it necessary, further dilutions i.e., 1/50 and 1/100 were performed). Then the sample is plated on appropriate agar media plates and incubated. Then the plate is examined for cfu and counted for total plate count. The samples were also speciated for cfu of E. coli, Salmonella, P. aeruginosa, S. aureus.

Testing of concentrations of 2-[2-(4-Chlorophenyl)ethoxy]adenosine in blood plasma: 2-[2-(4-Chlorophenyl)ethoxy]adenosine concentrations in blood plasma taken from a patient may be quantitated. A non-limiting example of an assay to quantitate 2-[2-(4-Chlorophenyl)ethoxy]adenosine in blood plasma is an HPLC/MS/MS bioanalytical assay. The assay used an organic precipitation of protein accomplished with addition of acetonitrile to plasma followed by a thorough mixing and centrifugation to separate the denatured protein and supernatant. Aliquots of the supernatant were injected onto a HPLC system equipped with a triple quadrapole mass spectrometer which provided highly specific and sensitive detection of the molecular ion of interest. Concentrations of 2-[2-(4-Chlorophenyl)ethoxy]adenosine were determined from peak area ratios of 2-[2-(4-Chlorophenyl)ethoxy]adenosine and the internal standard using weighted linear regression curves (1/x). The assay was highly robust and reproducible within the validated range of 1.0 to 100 ng/mL. There is considered to be no systemic absorption when the concentration of concentrations of 2-[2-(4-Chlorophenyl)ethoxy]adenosine in blood plasma falls below the LLOQ of the assay, which exhibited at LLOQ of 0.1 ng/mL in humans and 0.2 ng/mL in minipigs.

Testing for 2-[2-(4-Chlorophenyl)ethoxy]adenosine, as well as its degradant products, adenine, adenosine, isoguanosine and 2-(4-chlorophenyl)ethanol)in the formulations of the instant invention: The assay consisted of the use of an HPLC with the following operating conditions: Analytical column: Waters Atlantis dC-18, 5 μm, 250×4.6 mm ID; Temperature: 15° C.; Mobile Phase A: 100% Water; Mobile Phase B: 100% Acetonitrile; Flow Rate: 1.5 mL/min; Injection Volume: 100 μL; Detection: UV at 210 nm; Run Time: approx. 35 minutes. LOQ 2-[2-(4-Chlorophenyl)ethoxy]adenosine is: 0.004% drug substance alone; 0.004% for 5 μg/g gel; 0.003% for 500 μg/g. LOQ adenine is: 0.0007% drug substance alone; 0.005% for 5 μg/g gel; 0.002% for 500 μg/g. LOQ adenosine is: 0.002% drug substance alone; 0.004% for 5 μg/g gel; 0.003% for 500 μg/g. LOQ isoguanosine is: 0.001% drug substance alone; 0.02% for 5 μg/g gel; 0.003% for 500 μg/g.

Standards for adenine, adenosine, isoguanosine (10 mg). are prepared in acetonitrile (10 mL) and water (20 mL) and sonicated until dissolved dilute to a nominal concentration of 20 μg/mL. Standards and sample for 2-[2-(4-Chlorophenyl)ethoxy]adenosine were prepared by sonicating 10 mg of 2-[2-(4-Chlorophenyl)ethoxy]adenosine in 10 mL of acetonitrile and 20 mL of water and diluting with water until a nominal concentration of 20 μg/mL was reached. Samples for the pharmaceutical formulation gel were prepared by measuring 5 g of 5 μg/g gel or 4 g of 50 μg/g and 500 μg/g gel in to a 50 mL centrifuge tube. 7 mL of acetonitrile in 0.5 to 1 mL increments was added to the centrifuge tube, vortexing between additions. The carboxymethyl cellulose will precipitate out during the addition of acetonitrile. The mixture was then sonicated and centrifuged at 4800 rpm. The supernatant was decanted and collected in a scintillation vial along with the acetonitrile rinse (3×) of the pellet and tube. The collected liquid was dried (with increased temperature and warm air) for up to 3.5 hours. The remaining solution (along with the rinsate of the scintillation vial) was transferred to a flask and diluted with water until a nominal concentration of 20 μg/mL was reached.

The formula for the assay (% label claim) was: Assay=Rᵤ/Rₛ×C_{std}×D×1/W×100. Rᵤ is the peak area of 2-[2-(4-Chlorophenyl)ethoxy]adenosine (drug substance) in the sample, Rₛ is the average peak of all working standard A injections, C_{std} is the concentration of the working standard in μg/mL, including purity, D is the sample preparation dilution factor and W is the weight of sample in μg. If the assay is for the 2-[2-(4-Chlorophenyl)ethoxy]adenosine (for gel) the assay=Rᵤ/Rₛ×C_{std}×D×1/W×1/LC×100. Rᵤ is the peak area of 2-[2-(4-Chlorophenyl)ethoxy]adenosine (drug substance) in the sample, Rₛ is the average peak of all working standard A injections, C_{std} is the concentration of the working standard in μg/mL, including purity, D is the sample preparation dilution factor, W is the weight of sample in μg and LC is the label claim in μg/g. The assay for the percent of related compounds in the drug substance is=R_{RC}/Rₛ×C_{std}×D×1/W×100×RRF. R_{RC} is the peak area of the related compound in the sample, Rₛ is the average peak of all working standard A injections, C_{std} is the concentration of the working standard in c/mL, including purity, D is the sample preparation dilution factor, W is the weight of sample in μg and RRF is the relative response factor (adenine 0.55, adenosine 1.13, isoguanosine 1.14 and 2-(4-chlorophenyl)ethanol 1.84). For the percent of individual related compounds in the gels the assay is=R_{RC}/Rₛ×C_{std}×D×1/W×1/LC 100×RRF. R_{RC} is the peak area of the related compound in the sample, Rₛ is the average peak of all working standard A injections, C_{std} is the concentration of the working standard in μg/mL, including purity, D is the sample preparation dilution factor, LC is label claim μg/g and W is the weight of sample in μg and RRF is the relative response factor (adenine 0.55, adenosine 1.13, isoguanosine 1.14 and 2-(4-chlorophenyl)ethanol 1.84).

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

These examples are intended as preferred embodiments only, and are provided to further illustrate this invention. They are not intended, either individually, in combination, or collectively, to define the full scope of this invention.

### EXAMPLE 1

### Preparation of Pharmaceutical Composition

The following method was used to manufacture the compositions described below in Tables 2-4. Table 1, the placebo formulation, was made by the method listed below except that 2-[2-(4-Chlorophenyl)ethoxy]adenosine was not used.

Under ambient room temperature conditions, 2-[2-(4-Chlorophenyl)ethoxy]adenosine was dissolved in propylene glycol in a mixing vessel. The sodium carboxymethylcellulose was slowly added to propylene glycol mixture while stirring until lump-free. Purified water was added to a separate mixing vessel followed by the addition of the sodium acetate trihydrate, glacial acetic acid, and sodium chloride with mixing until dissolved. The purified water solution was slowly added to the propylene glycol mixture with mixing. The combined mixture was then homogenized and allowed to cool to room temperature. The resulting gel was filled into jars or tubes.

### Quantitative Formulations for 2-[2-(4-chlorophenyl)ethoxy]adenosine Gels

TABLE 1
Placebo Gel No active agent

| Material | % (w/w) | Amount (mg) |
|---|---|---|
| Propylene Glycol, USP | 50 | 500 |
| Sodium | 1.8 | 18 |
| Carboxymethylcellulose, USP | | |
| Sodium Acetate (trihydrate), | 0.15 | 1.5 |
| USP | | |
| Glacial Acetic Acid, USP | 0.01 | 0.1 |
| Sodium Chloride, USP | 0.78 | 7.8 |
| Purified Water | 47.26 | 472.6 |
| Total | 100 | 1000 |

TABLE 2
5 μg/g Gel

| Material | % (w/w) | Amount (mg) |
|---|---|---|
| 2-[2-(4- | 0.0005 | 0.005 |
| chlorophenyl)ethoxy]adenosine | | |
| Propylene Glycol, USP | 50 | 500 |
| Sodium | 1.8 | 18 |
| Carboxymethylcellulose, USP | | |
| Sodium Acetate (trihydrate), | 0.15 | 1.5 |
| USP | | |
| Glacial Acetic Acid, USP | 0.01 | 0.1 |
| Sodium Chloride, USP | 0.78 | 7.8 |
| Purified Water | 47.26 | 472.6 |
| Total | 100 | 1000 |

TABLE 3
50 μg/g Gel

| Material | % (w/w) | Amount (mg) |
|---|---|---|
| 2-[2-(4- | 0.005 | 0.05 |
| chlorophenyl)ethoxy]adenosine | | |
| Propylene Glycol, USP | 50 | 500 |
| Sodium Carboxymethylcellulose, | 1.8 | 18 |
| USP | | |
| Sodium Acetate (trihydrate), | 0.15 | 1.5 |
| USP | | |
| Glacial Acetic Acid, USP | 0.01 | 0.1 |
| Sodium Chloride, USP | 0.78 | 7.8 |
| Purified Water | 47.26 | 472.6 |
| Total | 100 | 1000 |

TABLE 4
500 μg/g Gel

| Material | % (w/w) | Amount (mg) |
|---|---|---|
| 2-[2-(4- | 0.05 | 0.5 |
| chlorophenyl)ethoxy]adenosine | | |
| Propylene Glycol, USP | 50 | 500 |
| Sodium | 1.8 | 18 |
| Carboxymethylcellulose, USP | | |
| Sodium Acetate (trihydrate), | 0.15 | 1.5 |
| USP | | |
| Glacial Acetic Acid, USP | 0.01 | 0.1 |
| Sodium Chloride, USP | 0.78 | 7.8 |
| Purified Water | 47.21 | 472.1 |
| Total | 100 | 1000 |

### EXAMPLE 2

### Antimicrobial (Self-Preserving) Properties of 2-[2-(4-Chlorophenyl)ethoxy]adenosine Formulations

This example showed that the inventive composition possesses antimicrobial properties which allow the composition to be prepared using non-aseptic methods yet maintain the formulation within set sterility and microbial limit levels (total count<10 cfu/g and yeasts and molds<10 cfu/g.)

Studies were conducted to examine the antimicrobial properties of formulations of the current invention as described in Example 1 for the 50, 500 μg/g and placebo formulations prepared under both aseptic and non-aseptic manufacturing conditions in a non-controlled laboratory setting and then packaging the formulation in pre-sterilized and non-sterilized laminate and aluminum tubes.

The aseptic manufacturing process was simulated by the use of a laminar flow hood and standard aseptic techniques. The non-aseptic manufacturing process was simulated by preparing the formulation at non-hooded laboratory bench at ambient conditions.

The packaging that was tested was pre-sterilized and non-sterilized tubes made of either laminate or aluminum. The pre-sterilized tubes were sterilized prior to use by placing them in containers and sterilizing them with gamma irradiation as listed in Table 5.

TABLE 5
Gamma Irradiation Exposure for Sterilized Tubes Used in
Packaging Study

| | Specified | | Delivered | | |
|---|---|---|---|---|---|
| | Dose (kGy) | | Dose (kGy) | | Exposure |
| Container | Minimum | Maximum | Minimum | Maximum | Time (min) |
| 1 | 25.0 | 45.0 | 30.4 | 36.5 | 278 |
| 2 | 25.0 | 45.0 | 30.4 | 35.7 | 277 |

Batches of placebo, 50 and 500 μg/g (2-[2-(4-Chlorophenyl)ethoxy]adenosine formulations were made as described in Example 1 under aseptic or non-aseptic conditions was pre-sterilized and non-sterilized tubes made of either laminate or aluminum were filled with each prepared formulation, sealed and place in controlled conditions of 25° C./60% RH for observation.

The 50 and 500 μg/g (2-[2-(4-Chlorophenyl)ethoxy]adenosine batch formulations as well as the placebo formulation prepared by the non-aseptic method were tested for microbial growth prior to filling of the tubes. They were tested for E. coli, P. aeruginosa, S. aureus, C. albicans and A. niger and showed <10 cfu/g. At five months the non-aseptic batch formulations were tested for sterility. The three non-aseptic batches were found to meet sterility requirements at five months. Results for both tests are found at Table 6.

TABLE 6
Microbiological Testing Results for Bulk Samples of Non-Aseptically
Prepared 2-[2-(4-chlorophenyl)ethoxy]adenosine Gels (Prior to Filling)

| | Microbial Limits (initial) | | |
|---|---|---|---|
| Strength | Total Plate Count | Yeasts and Molds | Sterility (5 months) |
| Placebo | <10 cfu/g | <10 cfu/g | Sterile |
| 50 μ/g | <10 cfu/g | <10 cfu/g | Sterile |
| 500 μg/g | <10 cfu/g | <10 cfu/g | Sterile |

A subset of samples from each of the packaging configurations encompassing ((20 each) for irradiated and non-irradiated aluminum and laminated tubes containing the three formulations (placebo, 50 and 500 μg/g) prepared by either aseptic methods or non-aseptic methods) were held for observation at 25° C./60% RH for 6 months. The compositions (placebo, 50 and 500 μg compositions) prepared with both non-aseptic and aseptic materials were subjected to Antimicrobial Effectiveness Testing as described in this application at 6 months. All tubes passed the Antimicrobial Effectiveness Testing specification at 14 and 28 days (for E. coli, P. aeruginosa, S. aureus, C. albicans. and A. niger.). In addition, the tubes were tested for sterility initially and sterility at three months S. aureus, Ps. Aeruginosa, C. sporogenes, B. subtili, C. albicans, A. niger.

At the initial sterility testing one positive growth in one of twenty samples was observed in the 500 μg/g non-aseptic formulation packaged in irradiated laminate tubes and two positive growths in one of twenty samples were observed the 50 μg/g non-aseptic gel formulation packaged in non-irradiated aluminum tubes. Both of the tubes that showed microbial growth at the initial testing did not show microbial at 3 months. The fact that positive growth was not seen in all four packaging configurations and the fact that these batches had been shown to be sterile prior to packaging suggest that the material was potentially contaminated during the filling process conducted in the non-controlled laboratory setting.

At 3 months, all batches and packaging configurations meet sterility requirements except for one 50 μg/g non aseptic gel tube out of 20 packaged in irradiated laminate tubes. This tube was tested at 6 months and passed AET testing (for E. coli, P. aeruginosa, S. aureus, C. albicans. and A. niger) at 6 months. The batches in which a positive growth was detected in the initial sterility testing were found to meet the sterility requirements at three months.

The results of these packaging studies also indicate that there is no difference in the microbiological quality of the gel when packaged in irradiated tubes versus non-irradiated tubes. Because the sterilized tubes do not offer additional microbiological protection to a product with innate antimicrobial activity, it is not necessary to use sterilized packaging for pharmaceutical gel compositions prepared according to the present invention.

### EXAMPLE 3

2-[2-(4-chlorophenyl)ethoxy]adenosine was formulated as described in Example 1 at 5, 50 and 500 μg/g concentrations. Additionally, a placebo formulation was produced as described in Example 1. The formulations were all prepared under non-aseptic methods. The three 2-[2-(4-chlorophenyl)ethoxy]adenosine formulations (5, 50 and 500 μg/g ) and the placebo formulation were placed in 0.5 oz (15 g) C39747 laminate tubes (Montebello, Inc., Hawkesbury, Ontario) sealed with tamper evident seals and No. 16 polypropylene Fez puncture cap. The three formulations and placebo were placed on stability testing at two different testing conditions, 25° C./60% RH and 40° C./75% RH. The formulation samples were tested initially at the commencement of the test and at regular internals for stability and microbial growth. The viscosity for the samples ranged from about 1,000,000 to about 1,600,000 cPs for the samples tested at either 25° C./60% RH for 12 months or 40° C./75% RH for 6 months.

The samples that were placed on stability testing were tested for 2-[2-(4-chlorophenyl)ethoxy]adenosine as well for its breakdown products adenine, adenosine, isoguanosine and 2-(4-chlorophenyl)ethanol). The assay, as additionally described in this application, consisted of the use of an HPLC with the following operating conditions: Analytical column: Waters Atlantis dC-18, 5 μm, 250×4.6 mm ID; Temperature: 15° C.; Mobile Phase A: 100% Water; Mobile Phase B: 100% Acetonitrile; Flow Rate: 1.5 mL/min; Injection Volume: 100 μL; Detection: UV at 210 nm; Run Time: approx. 35 minutes.

The results of the testing for 2-[2-(4-chlorophenyl)ethoxy]adenosine as well for its degradant products adenine, adenosine, isoguanosine and 2-(4-chlorophenyl)ethanol) as well as the total related compounds are shown in Tables 7-15. The placebo formulation was initially tested for 2-[2-(4-chlorophenyl)ethoxy]adenosine and was found to be absent. 2-[2-(4-chlorophenyl)ethoxy]adenosine is shown as % label.

TABLE 7
500 μg/g 25° C./60% RH

| | Initial | 1 month | 3 months | 6 months | 9 months | 12 months |
|---|---|---|---|---|---|---|
| 2-[2-(4- | 97 | 96 | 94 | 95 | 100 | 96 |
| chlorophenyl)ethoxy] | | | | | | |
| Adenosine | | | | | | |
| Adenine | ND | ND | ND | ND | ND | ND |
| Adenosine | ND | ND | ND | ND | ND | ND |
| Isoguanosine | ND | ND | ND | ND | ND | ND |
| 2-(4-chlorophenyl) | ND | ND | ND | ND | ND | ND |
| ethanol | | | | | | |
| Total related | 0.21 | 0.22 | 0.18 | 0.18 | 0.19 | 0.18 |
| compounds | | | | | | |

TABLE 8
50 μg/g 25° C./60% RH

| | Initial | 1 month | 3 months | 6 months | 9 months | 12 months |
|---|---|---|---|---|---|---|
| 2-[2-(4- | 97 | 97 | 93 | 98 | 102 | 99 |
| chlorophenyl)ethoxy] | | | | | | |
| adenosine | | | | | | |
| Adenine | ND | ND | ND | ND | ND | ND |
| Adenosine | ND | ND | ND | ND | ND | ND |
| Isoguanosine | ND | ND | ND | ND | ND | ND |
| 2-(4-chloro- | ND | ND | ND | ND | ND | ND |
| Phenyl) ethanol | | | | | | |
| Total related compounds | 0.20 | 0.21 | 0.18 | 0.19 | 0.19 | 0.18 |

TABLE 9
5 μg/g 25° C./60% RH

| | Initial | 1 month | 3 months | 6 months | 9 months | 12 months |
|---|---|---|---|---|---|---|
| 2-[2-(4- | 97 | 97 | 93 | 98 | 102 | 99 |
| chlorophenyl)ethoxy] | | | | | | |
| adenosine | | | | | | |
| Adenine | ND | ND | ND | ND | ND | ND |
| Adenosine | ND | ND | ND | ND | ND | ND |
| Isoguanosine | ND | ND | ND | ND | ND | ND |
| 2-(4-chloro- | ND | ND | ND | ND | ND | ND |
| Phenyl) ethanol | | | | | | |
| Total related compounds | ND | 0.09 | 0.13 | 0.20 | 0.19 | 0.16 |

TABLE 10
500 μg/g Gel 40° C./75% RH

| | Initial | 1 month | 3 months | 6 months |
|---|---|---|---|---|
| 2-[2-(4-chloro- | 97 | 96 | 89 (99) | 97 |
| phenyl)ethoxy] | | | | |
| adenosine | | | | |
| Adenine | ND | ND | ND | ND |
| Adenosine | ND | ND | ND | ND |
| Isoguanosine | ND | ND | ND | ND |
| 2-(4-chloro- | ND | ND | ND | ND |
| Phenyl) ethanol | | | | |
| Total related compounds | 0.21 | 0.22 | 0.18 | 0.18 |

TABLE 11
50 μg/g Gel 40° C./75% RH

| | Initial | 1 month | 3 months | 6 months |
|---|---|---|---|---|
| 2-[2-(4-chloro- | 97 | 98 | 96 | 99 |
| phenyl)ethoxy] | | | | |
| adenosine | | | | |
| Adenine | ND | ND | ND | ND |
| Adenosine | ND | ND | ND | ND |
| Isoguanosine | ND | ND | ND | ND |
| 2-(4-chloro- | ND | ND | ND | ND |
| Phenyl) ethanol | | | | |
| Total related compounds | 0.20 | 0.22 | 0.19 | 0.23 |

TABLE 12
5 μg/g Gel 40° C./75% RH

| | Initial | 1 month | 3 months | 6 months |
|---|---|---|---|---|
| 2-[2-(4-chloro- | 94 | 98 | 95 | 99 |
| phenyl)ethoxy] | | | | |
| adenosine | | | | |
| Adenine | ND | ND | ND | ND |
| Adenosine | ND | ND | ND | ND |
| Isoguanosine | ND | ND | ND | ND |
| 2-(4-chloro- | ND | ND | ND | ND |
| Phenyl) ethanol | | | | |
| Total related compounds | ND | 0.09 | 0.14 | 0.28 |

The samples were tested for microbial growth at the initiation of testing and at six and twelve months for total plate count and specifically for E. coli, Salmonella, S. aureus, Ps. Aeruginosa, as well as AET tested for E. coli, P. aeruginosa, S. aureus, C. albicans. and A. niger.

TABLE 13
Microbial Testing 500 μg/g Gel 25° C./60% RH

| | Initial | 6 Months | 12 Months |
|---|---|---|---|
| Total Plate Count | <10 cfu/g | <10 cfu/g | <10 cfu/g |
| Yeast and Molds | <10 cfu/g | <10 cfu/g | <10 cfu/g |
| E. coli | Absent | Absent | Absent |
| Salmonella | Absent | Absent | Absent |
| S. aureus | Absent | Absent | Absent |
| Ps. aeruginosa | Absent | Absent | Absent |
| Antimicrobial | Conforms | Conforms | Conforms |
| Effectiveness | | | |
| Test | | | |

TABLE 14
Microbial Testing 50 μg/g Gel 25° C./60% RH

| | Initial | 6 Months | 12 Months |
|---|---|---|---|
| Total Plate Count | <10 cfu/g | <10 cfu/g | <10 cfu/g |
| Yeast and Molds | <10 cfu/g | <10 cfu/g | <10 cfu/g |
| E. coli | Absent | Absent | Absent |
| Salmonella | Absent | Absent | Absent |
| S. aureus | Absent | Absent | Absent |
| Ps. aeruginosa | Absent | Absent | Absent |
| Antimicrobial | Conforms | Conforms | Conforms |
| Effectiveness | | | |
| Test | | | |

TABLE 15
Microbial Testing 5 μg/g Gel 25° C./60% RH

| | Initial | 6 Months | 12 Months |
|---|---|---|---|
| Total Plate Count | <10 cfu/g | <10 cfu/g | <10 cfu/g |
| Yeast and Molds | <10 cfu/g | <10 cfu/g | <10 cfu/g |
| E. coli | Absent | Absent | Absent |
| Salmonella | Absent | Absent | Absent |
| S. aureus | Absent | Absent | Absent |
| Ps. aeruginosa | Absent | Absent | Absent |
| Antimicrobial | Conforms | Conforms | Conforms |
| Effectiveness | | | |

TABLE 16
Microbial Testing Placebo Gel 25° C./60% RH

| | Initial | 6 Months | 12 Months |
|---|---|---|---|
| Total Plate Count | <10 cfu/g | <10 cfu/g | <10 cfu/g |
| Yeast and Molds | <10 cfu/g | <10 cfu/g | <10 cfu/g |
| E. coli | Absent | Absent | Absent |
| Salmonella | Absent | Absent | Absent |
| S. aureus | Absent | Absent | Absent |
| Ps. aeruginosa | Absent | Absent | Absent |
| Antimicrobial | Conforms | Conforms | Conforms |
| Effectiveness | | | |
| Test | | | |

### EXAMPLE 4

2-[2-(4-chlorophenyl)ethoxy]adenosine was formulated as described in Example 1 at 5, 50 and 500 μg/g concentrations. Additionally, a placebo formulation was produced as described in Example 1. The formulations were all prepared under non-aseptic methods. The three 2-[2-(4-chlorophenyl)ethoxy]adenosine formulations (5, 50 and 500 μg/g ) and the placebo formulation were placed glass jars (1 g fill) and sealed. The three formulations and placebo were placed on stability testing at 25° C./60% RH. The samples were placed upon stability testing for an extended period of time ranging from 1½ to 4½ years. At the end of the period it was found that a number of the samples had glass jar seals that were not well sealed and some of the samples seemed slightly dried. The samples were tested with the HPLC method as previously described for several selected known and total related compounds. The samples were not subjected to any microbiological testing. A 5 ug/g gel formulation (4½ years on stability) exhibited 2-[2-(4-chlorophenyl)ethoxy]adenosine at 107% of label claim, adenosine at 0.02%, isoguanosine at 0.19% and total related compounds at 4.71%. A 50 ug/g gel formulation (4½ years on stability) exhibited 2-[2-(4-chlorophenyl)ethoxy]adenosine at 133% of label claim, adenosine at 0.28%, isoguanosine at 0.15% and total related compounds at 1.29%. A 500 ug/g gel formulation (4½ years on stability) exhibited 2-[2-(4-chlorophenyl)ethoxy]adenosine at 116% of label claim, adenosine at 0.24%, isoguanosine at 0.12%, 2-(4-chlorophenyl)ethanol) at 0.02% and total related compounds at 1.20%. A 5 ug/g gel formulation (3 years 7 months on stability) exhibited 2-[2-(4-chlorophenyl)ethoxy]adenosine at 120% of label claim, adenosine at 0.23%, isoguanosine at 0.03% and total related compounds at 0.77%. A 50 ug/g gel formulation (2 years 8 months on stability) exhibited 2-[2-(4-chlorophenyl)ethoxy]adenosine at 116% of label claim, adenosine at 0.23%, isoguanosine at 0.15% and total related compounds at 1.21%. A 500 ug/g gel formulation (2 year 3 months on stability) exhibited 2-[2-(4-chlorophenyl)ethoxy]adenosine at 107% of label claim, adenosine at 0.22%, isoguanosine at 0.18% and total related compounds at 0.95%.

### EXAMPLE 5

### In vivo Tests With Non-Humans—Systemic Absorption

The objective of this study was to assess the local and the systemic toxicity of the 2-[2-(4-chlorophenyl)ethoxy]adenosine formulation of the instant invention. The testing occurred in Gottingen SPF minipigs. 20 μg/g, 100 μg/g and 500 μg/g concentration of the 2-[2-(4-chlorophenyl)ethoxy]adenosine formulation were created using the methods described in Example I. The 20 μg/g and 100 μg/g formulation are described below. The components of the 500 μg/g formulation and the placebo control were as described in Example 1.

TABLE 17
20 μg/g Gel

| | | Amount |
|---|---|---|
| Material | % (w/w) | (mg) |
| MRE0094 | 0.002 | 0.02 |
| Propylene Glycol, USP | 50 | 500 |
| Sodium | 1.8 | 18 |
| Carboxymethylcellulose, USP | | |
| Sodium Acetate (trihydrate), | 0.15 | 1.5 |
| USP | | |
| Glacial Acetic Acid, USP | 0.01 | 0.1 |
| Sodium Chloride, USP | 0.78 | 7.8 |
| Purified Water | 47.26 | 472.6 |
| Total | 100 | 1000 |

TABLE 18
100 μg/g Gel

| Material | % (w/w) | Amount (mg) |
|---|---|---|
| MRE0094 | 0.01 | 0.1 |
| Propylene Glycol, USP | 50 | 500 |
| Sodium | 1.8 | 18 |
| Carboxymethylcellulose, | | |
| USP | | |
| Sodium Acetate | 0.15 | 1.5 |
| (trihydrate), USP | | |
| Glacial Acetic Acid, USP | 0.01 | 0.1 |
| Sodium Chloride, USP | 0.78 | 7.8 |
| Purified Water | 47.25 | 472.5 |
| Total | 100 | 1000 |

The total dosage of 2-[2-(4-chlorophenyl)ethoxy]adenosine per minipig ranged from 80 μg to 2000 μg/day. The formulation was administered topically once per day in 1 mL (1 g) doses at 20 μg/g, 100 μg/g and 500 μg/g concentration per wound per administration on surgically established full-thickness wounds until wound closure. Each minipig had multiple (4) wounds that received treatment.

A total of 40 Göttingen SPF minipigs (20 males and 20 females) were included in the study. The animals were allocated into four groups each of 4 males and 4 females. In addition, 4 animals (2 of each sex per group), were included as recovery animals for the control/placebo group (Group 1) and the high-dose group (Group 4).

From Day 1 through Day 13, 2[2-(4-chlorophenyl)ethoxy]adenosine formulation at either 20 μg/g, 100 μg/g or 500 μg/g concentration was applied topically on surgically established circular full-thickness wounds (wound diameter 20 mm; 4 wounds per animal) on a daily basis in a dose volume of approximately 1 mL per wound until wound closure. The dose levels and animal numbers are provided Table 20.

TABLE 19
Topical Dose Levels and Animal Numbers

| | | |
|---|---|---|
| Group | Dose | Animal No. - Main Study |

| No | (μg/day) | Male | Female |
|---|---|---|---|
| 1 | Placebo | 1–4 | 5–8 |
| 2 | 80 | 9–12 | 13–16 |
| | (20 μg/g Gel) | | |
| 3 | 400 | 17–20 | 21–24 |
| | (100 μg/g Gel) | | |
| 4 | 2000 | 25–28 | 29–32 |
| | (500 μg/g Gel | | |

Toxicokinetic sampling occurred on Day 6 at the following time points: pre-treatment, and 0.5, 1, 3, 5, 7, 9, 12 and 24 hours post-treatment. All plasma samples collected from Group 1 control animals receiving placebo via topical showed 2-[2-(4-chlorophenyl)ethoxy]adenosine levels below the Lower Limit of Quantification (LLOQ) of 0.2 ng/mL with the exception of three samples when tested in an HPLC/MS/MS bioanalytical assay to quantitate 2-[2-(4-chlorophenyl)ethoxy]adenosine in blood plasma. Non-compartmental pharmacokinetic analysis of plasma 2-[2-(4-chlorophenyl)ethoxy]adenosine concentration profiles in Groups 2, 3, and 4 (low-, mid- and high-dose) animals yielded pertinent pharmacokinetic parameters, which are summarized by treatment and gender in Table 20.

TABLE 20
Topical Treatment 2-[2-(4-chlorophenyl)ethoxy]adenosine
Pharmacokinetic Parameters in Minipigs (Mean ± CV) on Day 6

| | Dose | Dose^{a} | | |
|---|---|---|---|---|
| Parameter | Group | (μg/day) | Female | Male |
| Cₘₐₓ (ng/mL) | 2 | 80 | ND^{c} | ND^{c} |
| | 3 | 400 | ND^{c} | ND^{c} |
| | 4^{b} | 2000 | 0.45 (47.7%) | 0.46 (48.4%) |
| Tₘₐₓ (hr) | 2 | 80 | ND^{c} | ND^{c} |
| | 3 | 400 | ND^{c} | ND^{c} |
| | 4^{b} | 2000 | 7.5 (31.3%) | 7.8 (30.7%) |
| T_{1/2} (hr) | 2 | 80 | ND^{c} | ND^{c} |
| | 3 | 400 | ND^{c} | ND^{c} |
| | 4^{b} | 2000 | 28.4 (46.4%) | 122 (218%) |
| AUC₂₄ₕ | 2 | 80 | ND^{c} | ND^{c} |
| (hr * ng/mL) | 3 | 400 | ND^{c} | ND^{c} |
| | 4^{b} | 2000 | 6.65 (52.9%) | 7.25 (60.6%) |

^{a}Administered via once daily topical application at 0 hr.
^{b}n = 6.
^{c}Pharmacokinetic parameters could not be determined because the majority of the plasma concentrations at the 80 and 400 μg/day topical dose were below the LLOQ of the assay.

During the wound healing phase, only the plasma concentration versus time profiles at the 2000 μg/day topical dose were analyzable to yield pharmacokinetic parameters. This finding showed that absorption into the systemic circulation from topical administration in open wounds was minimal. Following topical administration of the 2000 μg/day dose, the pharmacokinetic behavior of 2-[2-(4-chlorophenyl)ethoxy]adenosine in female and male minipigs was similar.

### EXAMPLE 6

### Wound Healing in Humans

2-[2-(4-Chlorophenyl)ethoxy]adenosine was administered to patients with chronic, neuropathic, diabetic foot ulcers (DFU). Patients 18-80 years old were randomized in a 1:3 ratio to standard DFU care plus vehicle gel (placebo formulation as described in Example 1), or standard DFU care plus a gel containing 2-[2-(4-chlorophenyl)ethoxy]adenosine prepared to the present invention (as described in Example 1). Standard care included routine sharp debridement, pressure offloading, and maintaining a moist wound environment. Inclusion criteria included cutaneous full thickness wounds between 1 and 10 cm² in area. Exclusion criteria included arterial insufficiency, renal or hepatic insufficiency, active infection, or osteomyelitis. Patients were enrolled into 3 groups, and received drug by group of 5 μg/g, 50 μg/g, or 500 μg/g. Drug or vehicle was applied topically once daily for 28 days. Outcome measures included adverse events and other safety assessments, plasma concentrations of the active agent (2-[2-(4-chlorophenyl)ethoxy]adenosine), percent of wound closed, and rate of wound closure.

Results: Thirty-six patients were randomized (25 active agent; 11 vehicle gel (placebo)) with an average age of 54.8 years; 78% were male. The patients were tested for systemic absorption of 2-[2-(4-chlorophenyl)ethoxy]adenosine initially and on day 16 and day 28. No systemic absorption of 2-[2-(4-chlorophenyl)ethoxy]adenosine was detected at any topical dose concentration (initial, day 16 and day 28) when blood plasma was tested using a HPLC/MS/MS assay (LLOQ 1.0 ng/mL) to detect the presence of 2-[2-(4-chlorophenyl) ethoxy]adenosine. The mean (±SD) wound size at randomization determined by planimetry was 0.91±0.63 cm² and did not differ between vehicle (placebo) and 2-[2-(4-chlorophenyl)ethoxy]adenosine groups. Percent wound closure at 28 days, and median days to 50% and 75% closure by treatment group are listed in Table 21 below.

TABLE 21

| | | % Wound | Median Number of Days to: | |
|---|---|---|---|---|
| Treatment | N | Closure (Day 28) | 50% Closure | 75% Closure |
| Vehicle | 11 | 33.3% | 22 | 37 |
| 5 μg/g | 7 | 60.7% | 8 | 14 |
| 50 μg/g | 9 | 67.5% | 12 | 28 |
| 500 μg/g | 9 | 35.4% | 14 | 36 |

### EXAMPLE 7

### Wound Healing in Humans

Multicenter, double-blind, randomized, parallel, vehicle-controlled, and standard care-controlled trials of topically applied 2-[2-(4-chlorophenyl)ethoxy]adenosine in diabetic subjects with chronic, neuropathic foot ulcers. A broad range of concentrations of the invention are studied in a wide variety of wound sizes (see Table 22 below) utilizing the formulations of Example 1. Approximately 340 subjects are enrolled in these studies. Approximately 300 subjects have wounds 1-5 cm is size and approximately 40 subjects have wounds >5 cm but ≦10 cm. Efficacy endpoints include the incidence of complete healing (complete epithelialization with no exudate) of the wounds, time to wound closure (days), and percent reduction in surface area of the wounds from baseline (before exposure to the invention) to various time points after exposure to the invention. Safety assessments include evaluating systemic exposure to topical 2-[2-(4-chlorophenyl)ethoxy]adenosine measured by plasma concentrations of 2-[2-(4-chlorophenyl)ethoxy]adenosine; adverse events, irritation scores, and other safety parameters routinely monitored in clinical trials. In general, subjects complete a 7-14-day Screening/Standard Care Run-in Period, a Treatment Period of up to 90 days, and a 28-day Post-treatment Period. All subjects receive standard care treatment for their wound(s) throughout the studies that is consistent with the American Diabetes Association Consensus Development Conference on Diabetic Wound Care (American Diabetes Association. Consensus development conference on diabetic foot wound care. Diabetes Care 1999; 22(8):1354-1360), and the clinical practice guidelines for diabetic foot disorders of the American College of Foot and Ankle Surgeons and the American College of Foot and Ankle Orthopedics and Medicine (Frykberg, et al., J Foot Ankle Surg 2000; 39(Suppl 5):S1-60).

TABLE 22
2-[2-(4-chlorophenyl)ethoxy]adenosine Gel Concentrations

| | 2-[2-(4-chlorophenyl)- |
|---|---|
| Concentration | ethoxy]adenosine μg/gram of Gel |
| 0.0005% | 5.0 |
| 0.005% | 50.0 |
| 0.05% | 500.0 |
| Vehicle (0%) | 0.0 |

All doses of study drug (active and vehicle-control gels) were/are provided by King Pharmaceuticals, Inc (Bristol, Tenn.)

It will be appreciated that, although specific embodiments of the invention have been described herein for purposes of illustration, various modifications may be made without departing from the spirit and scope of the invention. Accordingly, the invention is not limited except as by the appended claims. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims

What is claimed is:

1. A pharmaceutical composition comprising
a) an effective amount of a 2-alkoxyadenosine or 2-aralkoxyadenosine;
b) about 10 to about 70% w/w glycol; and
c) a thickening agent.

2. The pharmaceutical composition of claim 1, wherein the 2-alkoxyadenosine or 2-aralkoxyadenosine is a 2-aralkoxyadenosine.

3. The pharmaceutical composition of claim 1, wherein the 2-alkoxyadenosine or 2-aralkoxyadenosine is 2-[2-(4-chlorophenyl)ethoxy]adenosine.

4. The pharmaceutical composition of claim 3, wherein the amount of a 2-[2-(4-chlorophenyl)ethoxy]adenosine is about 0.00001 to about 0.10% w/w of the composition.

5. The pharmaceutical composition of claim 1, wherein the glycol is 40-60% (w/w) of the composition.

6. The pharmaceutical composition of claim 1, wherein the glycol is 50% (w/w) of the composition.

7. The pharmaceutical composition of claim 1, wherein the thickening agent is a cellulose.

8. The pharmaceutical composition of claim 7, wherein the cellulose is sodium carboxymethylcellulose.

9. The pharmaceutical composition of claim 1, further comprising an isotonic agent.

10. The pharmaceutical composition of claim 9, wherein the isotonic agent comprises a salt.

11. The pharmaceutical composition of claim 10, wherein the salt is sodium chloride.

12. The pharmaceutical composition of claim 1, further comprising water.

13. The pharmaceutical composition of claim 12, wherein the water is about 30% to about 90% w/w of the composition.

14. The pharmaceutical composition of claim 1, further comprising a buffering system.

15. The pharmaceutical composition of claim 1, wherein the pH of the composition is 4.5 to 11.0.

16. The pharmaceutical composition of claim 1, wherein additional preservatives are absent.

17. A pharmaceutical composition comprising 0.0005% w/w 2-[2-(4-chlorophenyl)ethoxy]adenosine, 50% w/w propylene glycol, 1.8% w/w sodium carboxymethylcellulose, 0.15% w/w sodium acetate (trihydrate), 0.01% w/w glacial acetic acid, 0.78% w/w sodium chloride and 47.26% w/w purified water.

18. A pharmaceutical composition comprising 0.005% w/w 2-[2-(4-chlorophenyl)ethoxy]adenosine, 50% w/w propylene glycol, 1.8% w/w sodium carboxymethylcellulose, 0.15% w/w sodium acetate (trihydrate), 0.01% w/w glacial acetic acid, 0.78% w/w sodium chloride and 47.26% w/w purified water.

19. A pharmaceutical composition comprising 0.05% w/w 2-[2-(4-chlorophenyl)ethoxy]adenosine, 50% w/w propylene glycol, 1.8% w/w sodium carboxymethylcellulose, 0.15% w/w sodium acetate (trihydrate), 0.01% w/w glacial acetic acid, 0.78% w/w sodium chloride and 47.21% w/w purified water.

20. A method of making the pharmaceutical composition of claim 1 comprising
a) combining the active agent with the vehicle to produce a solution, and
b) mixing the solution of step a) with a thickening agent,
to produce the pharmaceutical composition.

21. The method of claim 20, further comprising adding water to the product of step b).

22. The method of claim 20, further comprising adding an isotonic agent to the product of step b) to produce the pharmaceutical composition.

23. The method of claim 20, further comprising adding a buffer system to the product of step b) to produce the pharmaceutical composition.

24. A method of treating a mammal with a wound comprising administration of the pharmaceutical composition of claim 1.

25. The method of claim 24, wherein the mammal is human.

26. The method of claim 25, wherein the wound is a chronic wound.

27. The method of claim 26, wherein the chronic wound is a diabetic foot ulcer.

28. A kit for promoting wound healing comprising the pharmaceutical composition of claim 1 in an amount effective to promote wound healing.

29. The kit of claim 31 further comprising bandages, wound protective dressings, foams, sponges, pads, gauzes, collagen, film dressings, drapes or pastes.

30. The kit of claim 32, wherein the pharmaceutical composition impregnates the bandages, wound protective dressings, foams, sponges, pads, gauzes, collagen, film dressings, drapes or pastes.

31. A stabilized pharmaceutical composition comprising an effective amount of 2-[2-(4-chlorophenyl)ethoxy]adenosine where the pharmaceutical composition is stable for up to 36 months.

32. The composition of claim 31, wherein the stabilized pharmaceutical composition comprising an effective amount of 2-[2-(4-chlorophenyl)ethoxy]adenosine is stable for up to 24 months.

33. A pharmaceutical composition comprising an effective amount of 2-alkoxyadenosine or 2-aralkoxyadenosine wherein the 2-alkoxyadenosine or 2-aralkoxyadenosine is not systemically absorbed when administered to a patient.

34. A pharmaceutical composition comprising an effective amount of 2-[2-(4-chlorophenyl)ethoxy]adenosine wherein the 2-[2-(4-chlorophenyl)ethoxy]adenosine is not systemically absorbed when administered to a patient.

35. A pharmaceutical composition comprising an effective amount of 2-[2-(4-chlorophenyl)ethoxy]adenosine wherein the 2-[2-(4-chlorophenyl)ethoxy]adenosine is minimally systemically absorbed when administered to a patient.

36. The composition of claim 34, wherein the wherein the 2-[2-(4-chlorophenyl)ethoxy]adenosine is not systemically absorbed when administered to a patient as measured by levels of 2-[2-(4-chlorophenyl)ethoxy]adenosine in the blood plasma levels of the patient.

37. The composition of claim 1, wherein the pharmaceutical composition is self preserving for up to 36 months.

38. The composition of claim 1, wherein the pharmaceutical composition is self preserving for up to 24 months.

39. The composition of claim 1, pharmaceutical composition is self preserving for up to 12 months.

40. The composition of claim 1, wherein the pharmaceutical composition is antimicrobially effective for up to 36 months.

41. The composition of claim 1, wherein the pharmaceutical composition is antimicrobially effective for up to 24 months.

42. The composition of claim 1, pharmaceutical composition is antimicrobially effective for up to 12 months.
